(19) United States
(12) Patent Application Publication
(10) Pub. No.: US 2002/0102250 A1
(43) Pub. Date: Aug. 1, 2002

(54) Recombinant S. aureus peptide deformylase

(76) Inventors: Martin R. Deibel JR., Kalamazoo, MI (US); Anthony Y. Yem, Kalamazoo, MI (US); Cindy L. Wolfe, Portage, MI (US); Anne F. Vosters, Kalamazoo, MI (US)

Correspondence Address: MUETING, RAASCH & GEBHARDT, P.A., P.O. BOX 581415, MINNEAPOLIS, MN, 55458, US

(21) Appl. No.: 09/895,951

(22) Filed: Jun. 29, 2001

Related U.S. Application Data
(60) Provisional application No. 60/215,677, filed on Jun. 30, 2000.

(51) Int. Cl. A61K 38/46; C12N 9/52

(52) U.S. Cl. 424/94.6; 435/220

(57) ABSTRACT

An S. aureus peptide deformylase iron complex composition, a method of isolating the composition, and a method of using the composition to screen for compounds that interact with a peptide deformylase are provided. The composition of the invention retains greater than about 95% of its catalytic deformylase activity after being stored at a protein concentration of greater than about 10 mg/ml in 50% glycerol at −20° C. for six months.

This application claims the benefit of U.S. Provisional Application Serial No. 60/215,677, filed Jun. 30, 2000, which is incorporated herein by reference in its entirety.

### FIELD OF THE INVENTION

This invention relates to a stable composition comprising an iron complex of Staphylococcus aureus (S. aureus) peptide deformylase (pdf), a method of preparing the complex, and a method of screening compounds that react with S. aureus peptide deformylase.

### BACKGROUND

Peptide deformylase is a procaryotic enzyme that catalyzes the specific removal of the formyl group following translation and release from the ribosome. Following this step, methionine is usually removed in a concerted manner by methionine aminopeptidase. Both of these enzymes have been shown to be essential for survival in certain bacteria. In early studies reports circulated indicating that peptide deformylase was a very unstable enzyme when isolated from bacterial sources. Later when the recombinant enzyme became available several laboratories showed that E. coli enzyme could be produced as a functional enzyme with improved stability at the higher protein concentrations. However, this appeared to be true only for the zinc complex of E. coli peptide deformylase (originally believed to only be a zinc metalloproteinase). It was only later demonstrated that the active form is an iron containing metalloproteinase, and less importantly, that E. coli pdf is also capable of existing as a nickel coordinated form.

Since the Fe and Ni complexes have the highest reported catalytic activities, and assuming that Fe rather than Ni would be the complexed physiological form, most strategies have based inhibition upon the Fe-pdf complex. Unfortunately, there was no convenient way to produce the Fe complex of pdf, since it has been shown that the Fe complex has a very short half-life of around 30 seconds. Attempts at isolation had used an oxygen purging system consisting of glucose oxidase and catalase, although the method suffered from the problem that the addition of these other enzymes could potentially interfere with the outcome of most screening strategies.

### SUMMARY OF THE INVENTION

In one embodiment, the present invention provides S. aureus peptide deformylase iron complex compositions having an N-terminal and/or a C-terminal affinity tag. The compositions of the invention retain greater than about 95% of their catalytic deformylase activity after being stored at a protein concentration of greater than about 10 milligrams (mg) per milliliter (ml) in 50% glycerol at −20° C. for six months.

In another embodiment, the present invention provides isolated and purified S. aureus peptide deformylase iron complexes. Such complexes retain greater than about 95% of their catalytic deformylase activity after being stored at a protein concentration of greater than about 10 mg/ml in 50% glycerol at −20° C. for six months.

In another embodiment, the present invention provides methods of isolating stable S. aureus iron-liganded peptide deformylase complexes. Such methods include preparing a complex with an N-terminal or a C-terminal affinity tag and isolating the tagged complexes using immobilized-metal affinity chromatography or metal-chelate affinity chromatography.

In still another embodiment, the present invention provides a method of screening for compounds that interact with peptide deformylase. The method includes introducing the compound into a system that includes stable S. aureus iron-peptide deformylase complexes, and measuring for inhibition of peptide deformylase activity. Preferably the system is free of antioxidants, for example, catalase.

Definitions

An “isolated” polypeptide or polynucleotide means a polypeptide or polynucleotide that has been either removed from its natural environment, produced using recombinant techniques, or chemically or enzymatically synthesized. Preferably, a polypeptide or polynucleotide of this invention is purified, i.e., substantially free from other polypeptides and/or polynucleotides and associated cellular products.

“Substantially free” from other polypeptides and/or polynucleotides and associated cellular products means that the composition has less than about 2% by weight other polypeptides, and preferably less than about 1% by weight of other polypeptides.

“Stable” means that the composition retains greater than 95% of its catalytic deformylase activity after being stored at a protein concentration of greater than about 10 mg/ml in 50% glycerol at −20° C. for 6 months. Under assay conditions described below, the diluted enzyme retains nearly full catalytic activity of the Fe enzyme for up to 30 minutes. The reaction rate is linear in the absence of inhibitors (under conditions where less than 20% of the substrate is depleted) for that period of time.

Abbreviations

The following abbreviations are used throughout this disclosure:

Staphylococcus aureus (S. aureus)

Escherichia coli (E. coli)

Peptide deformylase (pdf)

β-nicotinamide adenine dinucleotide (NAD)

Oxidized β-nicotinamide adenine dinucleotide (NAD+)

Reduced β-nicotinamide adenine dinucleotide (NADH)

Isopropylthio-β-D-galactoside (IPTG)

The following abbreviations are used for amino acids throughout this disclosure:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

### BRIEF DESCRIPTION OF THE FIGURES

FIG. 1 depicts the result of sodium dodecyl sulfate gel electrophoresis of S. aureus peptide deformylase after the final purification step and after 10 concentration and storage in 50% glycerol to afford stabilization during storage at −20 C. Also shown (in lane 2) is a purified sample of Vibrio proteolyticus aminopeptidase used for the pdf assay.

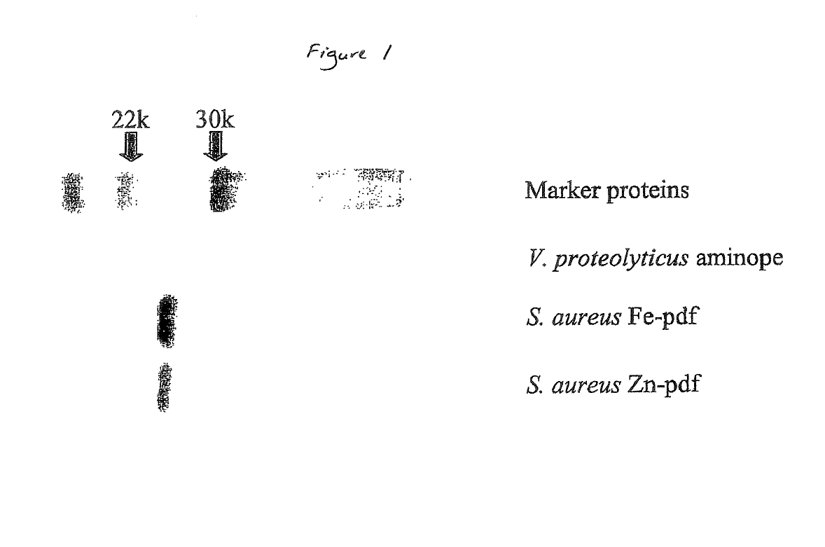

FIG. 2 illustrates the stability of S. aureus pdf at various dilutions by plotting optical density (OD; y-axis) vs. time (seconds; x-axis). FIG. 3(a) is a plot for an assay solution without added catalase. Lines are labeled for dilutions corresponding to (i) 562 nanograms (ng) (Vₘₐₓ₍ₐₚₚₐᵣₑₙₜ₎=165; R²=0.999); (ii) 281 ng (Vₘₐₓ₍ₐₚₚₐᵣₑₙₜ₎=81.8; R²=1.000); (iii) 140 ng (Vₘₐₓ₍ₐₚₚₐᵣₑₙₜ₎=41.1; R²=0.999); (iv) 70 ng (Vₘₐₓ₍ₐₚₚₐᵣₑₙₜ₎=20.5; R²=0.998); (v) 35 ng (Vₘₐₓ₍ₐₚₚₐᵣₑₙₜ₎=10.5; R²=0.997); and (vi) 17.5 ng (Vₘₐₓ₍ₐₚₚₐᵣₑₙₜ₎=6.3; R²=0.998). FIG. 3(b) is a plot for an assay solution with added catalase. Lines are labeled for dilutions corresponding to (i) 562 ng (Vₘₐₓ₍ₐₚₚₐᵣₑₙₜ₎=145; R²=0.998); (ii) 281 ng (Vₘₐₓ₍ₐₚₚₐᵣₑₙₜ₎=77.2; R²=1.000); (iii) 140 ng (Vₘₐₓ₍ₐₚₚₐᵣₑₙₜ₎=39.7; R²=0.999); (iv) 70 ng (Vₘₐₓ₍ₐₚₚₐᵣₑₙₜ₎=19.8; R²=0.998); (v) 35 ng (Vₘₐₓ₍ₐₚₚₐᵣₑₙₜ₎=10.1; R²=0.997); and (vi) 17.5 ng (Vₘₐₓ₍ₐₚₚₐᵣₑₙₜ₎=6.0; R²=0.999).

FIG. 3 illustrates the stability of E. coli pdf at various dilutions by plotting optical density (OD; y-axis) vs. time (seconds; x-axis). FIG. 4(a) is a plot for an assay solution without added catalase. Lines are labeled for dilutions corresponding to (i) 1000 ng (Vₘₐₓ=18.9; R²=1.000); (ii) 500 ng (Vₘₐₓ=6.5; R²=1.000); (iii) 250 ng (Vₘₐₓ=3.7; R²=0.999); and (iv) 125 ng (Vₘₐₓ=2.4; R²=0.999). FIG. 4(b) is a plot for an assay solution with added catalase. Lines are labeled for dilutions corresponding to (i) 1000 ng (Vₘₐₓ=133; R²=1.000); (ii) 500 ng (Vₘₐₓ=45.1; R²=1.000); (iii) 250 ng (Vₘₐₓ=19.2; R²=1.000); and (iv) 125 ng (Vₘₐₓ=8.4; R²=1.000).

FIG. 4 illustrates the effect of metal supplementation on the specific activity of Ni-NTA purified S. aureus pdf protein by plotting specific activity values (mOD/minute; y-axis) vs. concentration (nanograms; x-axis). Points and the best fit lines by least squares regression (m=slope; b=y-intercept; R²=square of the correlation coefficient) were plotted for pdf generated from media enriched with iron (points designated by “∘”; line A with m=0.096, b=0.887, and R²=0.997); pdf generated from media enriched with zinc (points designated by “□”; line B with m=0.015, b=0.604, and R²=0.998); and pdf generated from unsupplemented media (points designated by “ [symbol] ”; line C with m=0.024, b=0.589, and R²=0.999).

### DETAILED DESCRIPTION OF PREFERRED EMBODIMENTS

S. aureus peptide deformylase may be expressed in E. coli, and the protein purified to homogeneity by immobilized-metal affinity chromatography (IMAC) using nickel-iminodiacetic acid resin Ni-IDA), or by for metal-chelate affinity chromatography using nickel-nitrilotriacetic acid resin (Ni-NTA). Additional resolution of active and inactive forms of peptide deformylase may be obtained by ion exchange chromatography. The active fraction may be analyzed by several assay protocols and shown to be catalytically active as a deformylase. Assay systems have been developed as the basis for systems to screen compounds for interaction with a peptide deformylase by measuring for inhibition of peptide deformylase activity.

Affinity Tags

Affinity tags are residues that may be incorporated into a protein to enable isolation of the protein by immobilized-metal affinity chromatography or metal-chelate affinity chromatography. Examples of affinity tags useful in the isolation of proteins include, but are not limited to, His-tags (consecutively connected histidine units), glutathione S-transferase, Maltose binding protein, FLAG-tag (monoclonal FLAG antibodies and matrices, commercially available from BAbCO (Richmond, Calif.); may be introduced into the protein by primer design for PCR), and thioredoxin (a fusion protein system that can be captured by an anti-thioredoxin antibody column; vector available from Invitrogen Corp.; Carlsbad, Calif.). Useful His-tags include, but are not limited to, nxHis (where n is the number of consecutively connected histidine units), wherein n=4-8, and mixtures thereof. Particularly useful His-tags include small size tags (having a molecular weight of no greater than about 1 kDa) such as 6xHis (0.84 KDa, available from Qiagen, Inc., Valencia, Calif.), and those created via the polymerase chain reaction (PCR). Other useful small size tags include FLAG, and other peptides for which appropriate commercial antibodies are available for the purification of the fusion protein. Affinity tags are commonly placed on the N-terminus of the desired protein. However, small size affinity tags may be placed on the C-terminus, the N-terminus, and/or internally for the desired protein.

Affinity tags may be incorporated into the protein using, for example, commercially available expression systems such as that available under the trade designation QIAexpress Expression System available from Qiagen, Inc., Valencia, Calif. Commercially available vectors (Qiagen, Inc.) offer the possibility of placing the 6xHis tag at either the N-terminus and/or the C-terminus of the recombinant protein.

Isolation and Purification Procedures

The cell paste produced may be lysed in a buffer solution (e.g., Tris.HCl pH 8.0) using, for example, an enzyme like lysozyme at a temperature of about 2° C. to about 8° C. for about 10 minutes to about 15 minutes. The lysed material may be clarified by centrifugation, and then mixed with a chromatographic resin. For example, when a 6xHis tag is used, the tagged material may be readily isolated by immobilized-metal affinity chromatography using Ni-NTA resin. When other tags are used (e.g., GST, FLAG, etc.) other chromatographic resins (e.g., glutathione Sepharose, anti-FLAG IgG Sepharose, etc.) with an affinity for the tagged composition may also be used to separate the tagged material. The bound material is then washed with buffer, followed by elution with the appropriate solvent (e.g., imadazole/buffer mixture for Ni-NTA, glutathione/buffer for glutathione Sepharose, FLAG peptide for anti-FLAG IgG Sepharose, etc.). The eluted material may be concentrated by ultrafiltration using an appropriate membrane (e.g, YM10, PM10, YM3, etc. from Amicon; Bedford, Mass.). The isolation procedure is rapid, with the isolation of the lysed material being completed in less than about 5 minutes. After isolation, the purification procedure is generally completed in a period of about 2 hours to about 3 hours or more. Ion exchange chromatography using resins such as Q fast flow (Amersham Pharmacia Biotech, Inc.; Piscataway, N.J.), may be used to purify the isolated composition. A NaCl gradient may be used to resolve the less active or inactive forms of pdf.

Sodium dodecylsulfate polyacrylamide-gel electrophoresis (SDS-PAGE) and Western blotting techniques may be used for detecting and monitoring the state of purification of S. aureus peptide deformylase. The proteins may be visualized by staining with, for example, Coomassie blue or Ponceau S.

Assays for Analysis of S. aureus Peptide Deformylase

Direct and/or coupled assays may be used to analyze purified S. aureus peptide deformylase. A solution of S.aureus peptide deformylase and a reagent that reacts with S. aureus peptide deformylase is prepared, and reaction products are produced. A direct assay is a procedure in which the concentration of one or more of the reaction products is determined directly by analytical methods such as high pressure liquid chromatography (HPLC). A coupled assay is a procedure in which the concentration of the reaction product is determined indirectly by the subsequent addition of a second reagent that reacts with the first reaction product to produce a second reaction product. The second or coupled reaction product is then analyzed either directly, or indirectly through further coupled reactions.

### EXAMPLES

The present invention is illustrated by the following examples. It is to be understood that the particular examples, materials, amounts, and procedures are to be interpreted broadly in accordance with the scope and spirit of the invention as set forth herein.

### Materials

K-casein was from Sigma Chemical Co. (St. Louis, Mo.). The appropriate cDNA clone coding for S. aureus peptide deformylase in E. coli strain K12 (pTac-pdf-M2) was obtained using standard procedures in collaboration with Human Genome Sciences.

### Methods

Expression of Peptide Deformylase.

S. aureus peptide deformylase protein with a C-terminal 6xHis tag was expressed in E. coli strain K12 using Luria Broth (LB) with ampicillin added at 100 mg/L as the seed and production media. LB was prepared from Bacto tryptone (10 g), Bacto yeast extract (5 g), and NaCl (5 g), all amounts added per liter (L) of deionized water. The presterilization pH was adjusted to 7.5 with KOH and LB was autoclaved for 20 minutes in 100 ml volumes contained in 500 ml wide mouth fermentation flasks. Filter sterilized ampicillin was added to sterile LB at a final concentration of 100 mg/L just before inoculation. The 100 ml seed fermentations were carried out in the 500 ml wide mouth flasks, were inoculated using agar cultures or frozen stocks, and were incubated overnight at 37° C. with agitation at 200 revolutions per minute (rpm). The overnight seed fermentations were used to inoculate (2% rate) the 100 ml production fermentations carried out in 500 ml wide mouth flasks. The inoculated production fermentations were incubated at 37° C. with agitation at 200 rpm for slightly longer than 2 hours, at which time the optical density (OD) at 660 nanometers (nm) reached about 0.6. At this time, isopropylthio-β-D-galactoside (IPTG) was added to a final concentration of 0.4 millimolar (mM). The induced fermentations were continued as described for another 3.5 hours until the OD at 660 nm reached about 3.0. Four to 6 L fermentation volumes were achieved using multiple fermentation flasks, and harvest was by centrifugation.

For the above procedure, the iron form of pdf was the predominant product after purification. The Zn-enriched form of the enzyme was prepared using M9 glucose production medium containing thiamin, ZnCl₂, and ampicillin at 3, 20, and 100 mg/L, respectively. This medium was inoculated at a 10% rate using a seed culture prepared in the same medium except lacking Zn, and was fermented and induced as described for the preparation of the iron containing enzyme. Harvest was carried out as described previously at 3 hour post-induction when the OD reached about 1.3 units.

Ni-IMAC Chromatography For Purification of the Iron and Zinc Forms of S. aureus Pdf.

Cell paste from E. coli expressing S. aureus pdf (2-4 liter batches) was lysed in 150 milliliters (ml) of 50 mM Tris-HCl, pH 8.0, with lysozyme (Sigma Chemical Co.; St. Louis, Mo.) dissolved at 1 mg/ml. The suspension was allowed to stand on ice for approximately 10 minutes. The release of high molecular weight DNA resulting from cell lysis was partially degraded by shearing (repeated loading and reloading of a 10 ml syringe fitted with a 19 guage needle) to help reduce viscosity. The cell extract was collected and clarified by centrifugation (SS-34 rotor, Sorvall from Kendro Laboratory Products; Newtown, Conn.) at 20,500 rpm for 40-45 minutes at 5° C. Ten to fifteen milliliters of packed Ni-NTA resin (Qiagen, Inc., Valencia, Calif.), equilibrated in lysis buffer (without lysozyme) as described above, was stirred into the cell extract. This suspension was poured into a column and washed extensively with buffer as described above. His-tagged pdf protein was affinity eluted with 200 mM imidazole solution (approximately 50 ml) prepared in the lysis buffer mentioned above. This eluate was concentrated by ultrafiltration with a stirred cell (50 ml from Amicon; Bedford, Mass.) under nitrogen at room temperature to a final volume of approximately 3 ml.

Ion Exchange (Q Fast Flow) Chromatography.

The Fe²⁺ form of S. aureus pdf is prone to oxidation as is the case for the E. coli form of the enzyme (Rajagopalan et al., Biochemistry, 36:13910-18 (1997); Rajagopalan et al., J. Am. Chem. Soc., 19:12418-19 (1997)). However, there was less of an observed effect on the S. aureus pdf activity than previously reported for the E. coli enzyme. The concentrated protein sample was injected onto a 1 ml column (HR 1/5 from Amersham Pharmacia Biotech, Inc.; Piscataway, N.J.) packed with resin and equilibrated with 25 mM Tris.HCl, pH 8.0. Proteins were resolved with a linear gradient of sodium chloride. Protein eluted in the early part of the gradient was collected and further concentrated in a stirred cell (10 ml from Amicon; Bedford, Mass.) to a desired volume. For storage, the purified protein solution was adjusted to approximately 50% glycerol (v/v), mixed well, and stored at −20° C. The excellent stability of the iron form of S. aureus pdf, as compared to the less stable form of E. coli pdf (Fe), had been demonstrated by repeated enzyme assays over a year in time. The S. aureus Zn-pdf bound tighter to the Q fast flow matrix then that described for the Fe-pdf. However, the resolution of the two metal species of pdf in the mixture was not achieved, leaving a cross contamination of one metal form with the other form. But, since the kcat's differ significantly, interpretation of the data was not a problem.

Determination of Iron Content in Fe²⁺ Pdf.

Documentation of the presence of iron in the pdf enzyme preparation was made by Graphite Furnace Atomic Absorption Spectroscopy (GFAAS). The sample of pdf that was submitted for analysis was shown to consist predominantly of iron and zinc, although the presence of other metals at low levels could not be ruled out.

Electrophoresis and Western Blotting.

SDS gel electrophoresis was performed as described previously (Laemmli, Nature, 227:680-85 (1970)). SDS polyacrylamide gel electrophoresis was performed in 10% polyacrylamide gel and blotted onto polyvinylidene fluoride (PVDF) matrices with a semi-dry blotter at 125 milliamps (mA) for 8 minutes. Blots were stained with Coomassie blue R250 and destained in 50% ethanol and air dried for record keeping and storage.

Sequence Validation.

The sequence of S. aureus pdf was authenticated by N-terminal sequence analysis and by mass spectroscopy.

Preparation of Vibrio Proteolyticus Aminopeptidase.

The Vibrio proteolyticus (formerly called Aeromonas proteolytica) strain was purchased from Dr. Dehua Pei at Ohio State University, and was deposited in the Kalamazoo site culture collection as UC 15166. Since this organism is a marine bacterium, ocean salts were incorporated into all growth media. Stocks of the organism were prepared from 16 hour cultures grown in Difco Marine Broth (BD Biosciences; Sparks, Md.) with 20% glycerol added as a cryopreservative and were stored in liquid nitrogen vapor phase.

For production of aminopeptidase, the process described in Methods in Enzymology (Prescott et al., Methods in Enzymology, 45B:530-43 (1976)) was utilized with some modifications. A 0.1 ml aliquot of the glycerol stock culture was inoculated into 100 ml of seed medium contained in a 500 ml wide-mouth flask. The seed medium was composed of Sea Salt (20 g, Kent Marine, Inc.; Acworth, Ga.) and Bacto-Peptone (5 g), all amounts added per liter of de-ionized water. The culture was incubated at 28° C. for 8 hours on a rotary shaker at 250 rpm. The production medium was inoculated with 1% of the 8 hour seed culture and was grown as above, except the temperature was reduced to 26° C. The production medium consisted of enzymatic hydrolysate of casein from bovine milk (20 g of NZ-Amine AS from Sigma Chemical Co.; St. Louis, Mo.) and Sea Salt (26.4 g, Kent Marine, Inc.; Acworth, Ga.), all amounts per liter of deionized water. The pH was adjusted to 8.3 with concentrated KOH. After autoclaving, filter sterilized K₂HPO₄ and ZnCl₂ were added to final concentrations of 100 mg/l and 30 mg/l respectively. The culture was harvested after 16 hours and cells were removed from the fermentation by centrifugation at 6,000× g for 45 minutes. The supernatant was kept refrigerated until processed.

The enzyme was purified in a manner similar to that described in Chen et al., Biochemistry, 36:4278-86 (1997), and included an octyl Sepharose step. The final Q Sepharose fast flow column step was not utilized for our workup. By SDS-PAGE, a single band was observed at the appropriate molecular weight. The resulting preparation, generally concentrated by ultrafiltration (Amicon; Bedford, Mass.) to greater than 2.5 mg/ml, was stored in 50% glycerol at −20° C.

Assay Development For Peptide Deformylase.

Several assays were run to analyze the purified recombinant S. aureus peptide deformylase. These assays were based in part on the methods in Wei et al., Anal. Biochem., 250:29-34 (1997); and Lazennac et al., Anal. Biochem., 244:180-82 (1997). Assay conditions are described as detailed below:

(I). Pdf Coupled Assay with V. Proteolytica Aminopeptidase.

A description of the optimized assay conditions is as follows. The final assay solution contained 50 mM Hepes, 10 mM NaCl, pH 7.0, 0.1% K-casein (Sigma Chemical Co.; St. Louis, Mo.), 100 micromolar formyl-Met-Leu-p-nitroanilide (prepared by standard methods and commercially available from Bachem Bioscience, Inc.; Philadelphia, Pa.; Catalog No. L2030), 5 nanograms of pdf protein (iron form) or 100 nanograms of pdf protein (zinc form), and V. proteolytica aminopeptidase.

To set up the assay, the following steps were taken:

(A) 25 microliters of inhibitor in 15% dimethyl sulfoxide (DMSO) (maximum) was pipetted into 96 well microtiter plates (Immulon II from Dynex Technologies; Chantilly, Va.).

(B) 100 microliters of bulk assay solution was pipetted next. This solution is described as follows, and required that the reagents be mixed in the order shown below (enough for 100 assays):

TABLE 1
Stock Assay Solution

| | | Fe-pdf | Zn-pdf |
|---|---|---|---|
| | | Bulk Assay | Bulk Assay |
| | Preliminary | Solution for 1 | Solution for 1 |
| Reagents | preparation | 96 well plate | 96 well plate |
| 0.1% K-casein/50 | None | 11,376 | 11,376 |
| mM Hepes/10 mM | | microliters | microliters |
| NaCl, pH 7.0 | | | |
| Peptide deformylase | Dilute 1:100 into | | 600 |
| (0.1 mg/ml) (Fe | assay buffer (see | | microliters |
| enriched form) | above) | | |
| Peptide deformylase | Dilute 1:5 into | 600 | |
| (0.1 mg/ml) ) (Zn | assay buffer (see | microliters | |
| enriched form) | above) | | |
| Vibrio | None | 24 | 24 |
| aminopeptidase | | microliters | microliters |
| stock (3.9 mg/ml) | | | |
| Total Volume | | 12,000 | 12,000 |
| | | microliters | microliters |

The aminopeptidase concentration used here was in large excess. We found that one tenth of this amount was sufficient to keep the pdf reaction as the rate limiting step.

(C) The plate was preincubated at room temperature for 10 minutes.

(D) Finally, 25 microliters of 0.6 mM f-Met-Leu-p-nitroanilide (prepared by adding 120 microliters of 20 mM stock in 100% MeOH to 3,880 microliters of assay buffer) was added. Sometimes we found that the diluted substrate would be cloudy, a condition rapidly changed by incubation at 37° C.

(E) Each plate was examined in a SpectraMax 250 reader (Molecular Devices Corp.; Sunnyvale, Calif.) using an absorbance of 405 nm. Time points were taken every 20 seconds over a 30 minute time course at 25° C. Since oxidation might be an issue, the plate was mixed to begin the reaction, but not prior to subsequent time points. Addition of anti-oxidant reagents is not required, and preferably reducing agents are not added. Each line was checked for linearity over time and the resulting R² value was expected to be greater than about 0.98 for assay validity.

(II). Pdf Coupled Assay with Pseudomonas Oxalaticus Formate Dehydrogenase (fdh, F-9166 From Sigma Chemical Co.; St. Louis, Mo.).

A description of the optimized assay conditions are as follows. The final assay solution contained 50 mM Hepes, 10 mM NaCl, pH 7.0, 0.1% gelatin (EIA grade from Bio-Rad Laboratories; Hercules, Calif.), 250 micromolar formyl-Met-Ala-Ser, 20-80 nanograms of pdf protein, 3.125 mM NAD+, and 5-50 micrograms (μg) of formate dehydrogenase. The reagents, in the absence of pdf, could be mixed together in advance and stored on ice. However, since the assays were usually run with only a few samples, often the reagents were added individually. No problems seemed to occur as a result of reagent addition order. To set up the assay, the following steps were taken:

TABLE 2
Stock Assay Solution

| | | Fe-pdf | Zn-pdf |
|---|---|---|---|
| | | Bulk Assay | Bulk Assay |
| | Preliminary | Solution for 1 | Solution for 1 |
| Reagents | preparation | 96 well plate | 96 well plate |
| 0.1% K-casein/50 | None | 5,250 | 6,750 |
| mM Hepes/10 mM | | microliters | microliters |
| NaCl, pH 7.0 | | | |
| Peptide deformylase | Dilute 1:100 into | 3,000 | none |
| (0.1 mg/ml) | assay buffer (see | microliters | |
| | above) | | |
| Peptide deformylase | Dilute 1:100 into | none | 1,500 |
| (0.1 mg/ml) | assay buffer (see | | microliters |
| | above) | | |
| 20 mM NAD+, | None | 3,000 | 3,000 |
| pH 7.0 | | microliters | microliters |
| Formate dehydro- | | 750 | 750 |
| genase (5 mg/ml) | | microliters | microliters |
| Total Volume | | 12,000 | 12,000 |
| | | microliters | microliters |

(A) 25 microliters of inhibitor in 15% DMSO (maximum) or control with no inhibitor was pipetted into 96 well microtiter plates (UV plates, 96 well, #3635 from Costar; High Wycombe, Bucks, U.K.).

(B) 100 microliters of bulk assay mixture (above) was added.

(C) The plate was pre-incubated at room temperature for 10 minutes.

(D) 25 microliters of 5 mM f-Met-Ala-Ser (Bachem Bioscience, Inc.; Philadelphia, PAa. (in assay buffer) was added to start the reactions.

Each plate was examined in a SpectraMax 250 reader (Molecular Devices Corp.; Sunnyvale, Calif.) using an absorbance of 340 nm. Time points were taken every 20 seconds over a 30 minute time course at 25° C. Since oxidation might have been an issue, the plate was mixed to begin the reaction, but not prior to subsequent time points. Each generated line was checked for linearity over assay time and the resulting R² value was expected to be greater than about 0.98 for assay validity.

(III). Pdf Direct Assay Using HPLC Retention Time Readout.

The assay methods described above relied on coupling to the aminopeptidase or to fdh to generate a colorimetric assay (p-nitroaniline) which was followed at 405 nm, or a UV assay (NADH, reduced nicotinamide adenine dinucleotide) which was followed at 340 nm. An HPLC end point assay is also needed to validate inhibitors where both of the two assays do not resolve specific and non-specific inhibition. For the HPLC assay, we elected to follow the generation of the product Met-Leu-p-nitroanilide described in the reaction above. By tracking its UV absorbance at 315 nm and identifying the retention time on a C 18 reverse phase column using an acetonitrile gradient, the substrate could be easily distinguished from the product. Assays were set up in Eppendorf tubes (1.5 ml) or sample vial. 150 microliters of buffer (50 mM Tris.HCl, pH 7.5, 150 mM NaCl) was added along with 5 microliters of compound solubilized in 100% DMSO. Pdf was prepared by dilution of the stock enzyme solution (50% glycerol) in the assay buffer, and the solution was mixed and incubated at room temperature for 10 minutes. Substrate, fML-pNA, was first solubilized in 95% ethanol to give a 33 mM solution, and then 5 microliters was added to the assay solution. This solution was incubated at room temperature for 5 minutes. To terminate the reaction, an equal volume of 0.1% trifluoroacetic acid (TFA) was added. The acidified solution was analyzed by C18 reverse phase chromatography with a linear gradient (28 to 80% acetonitrile in 0.1% TFA over a period of 10 minutes). The UV monitor wavelength was set at 315 nm to detect the p-nitroaniline moiety of the substrate or product. The activity of the inhibitor was roughly quantified by peak height of the product in comparison to that of a control assay (substrate alone, and without the added inhibitor).

Stability of the Iron Form of S. aureus Peptide Deformylase.

Expression of the iron form of S. aureus or E. coli pdf (known to be unstable) in E. coli was monitored after the addition of 1.6 mM FeCl₃ in the media (Luria broth). Incubation was allowed to proceed for 1 hour prior to the addition of IPTG (0.4 mM). Induction continued for 3 hours at 37° C. Cells were isolated and extracted in the presence of catalase (10 micrograms/ml). The purpose of the catalase was to act as an oxygen scavenger to prevent the inactivation of the Fe form of the E. coil pdf. The extracts were subjected to Ni-NTA chromatography for the purification of the C-terminally His tagged versions of both the E. coli and S. aureus pdf proteins. The purified proteins (concentrated to at least 1 mg/ml by ultrafiltration using a membrane available from Amicon; Bedford, Mass.) were stored in the presence of 10 micrograms/ml (minimum concentration) catalase. To examine the stability of the Fe forms of the pdf proteins, dilutions of the proteins were made in the presence or absence of catalase, followed by assays of the enzymes (serially diluted) in the presence or absence of catalase (at 100 micrograms/ml). Assays were run using formyl-Met-Leu-p-nitroanilide as substrate, coupled to the Vibrio aminopeptidase.

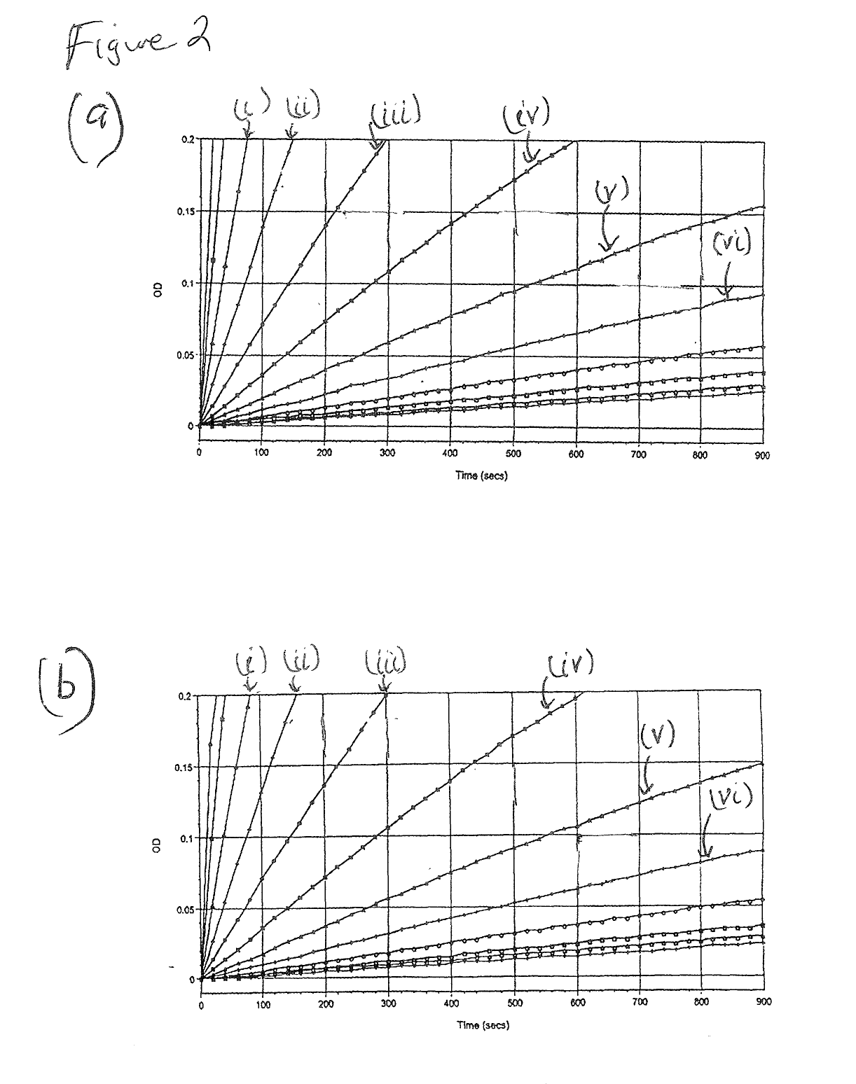

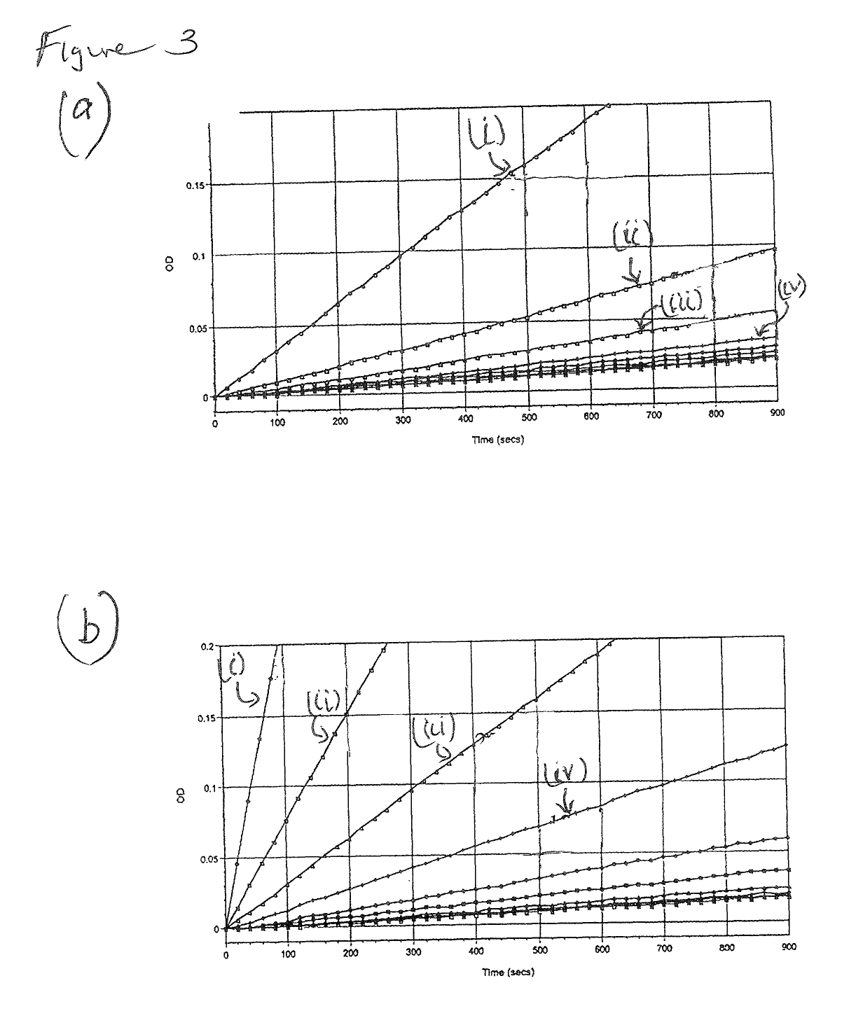

The results, which depict linear reactions catalyzed by pdf, are shown in FIG. 2 for S. aureus pdf and FIG. 3 for E. coli pdf. Clearly, the E. coli Fe-pdf enzyme rapidly loses activity (measured as changes in the rate of hydrolysis of the substrate, formyl-Met-Leu-p-nitroanilide using the Vibrio aminopeptidase coupled assay method). The rates shown are in mOD/minute, and each line represents a different concentration of the pdf in nanogram amounts as listed in the legend. For S. aureus pdf, the rates of pdf activity as a function of dilution of the pdf protein are almost identical, whether or not catalase is added to the assay solution, while the rates for the E. coil enzyme are drastically different under these conditions. This is consistent with a significant loss of enzyme activity in the absence of catalase only for the E. coli enzyme. See Rajagopalan et al., J. Amer. Chem. Soc., 119:12418-19 (1997).

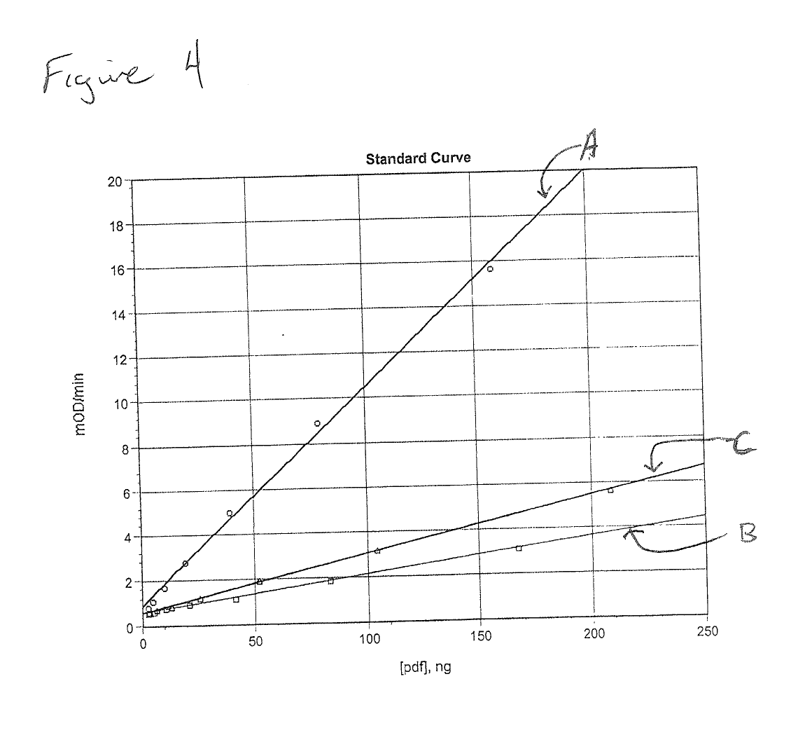

As stated above, the stable Fe form of S. aureus pdf (6xHis) can be prepared by supplementation of FeCl₃ in the media during induction. Similarly, one can prepare the Zn form of the enzyme by supplementation with ZnCl₂ (the specific activity (defined as measured rates of activity in the defined assay per unit concentration of protein) of the Zn form of pdf is much lower than that of the Fe form). After such treatment the enzyme is extracted and purified by Ni-NTA chromatography as described above (in the absence of catalase). The pdf from each experiment (Fe, Zn, or no supplementation) was then assayed as shown in FIG. 4. The addition of Zn lowers the specific activity of the pdf (compared to the control media), while the addition of Fe increases the specific activity by 3-6 fold (average) (compared to control media).

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An S. aureus peptide deformylase composition comprising an iron complex having an N-terminal and/or a C-terminal affinity tag, wherein the composition retains greater than about 95% of its catalytic deformylase activity after being stored at a protein concentration of greater than about 10 mg/ml in 50% glycerol at −20° C. for six months.

2. The composition of claim 1, wherein the affinity tag is selected from the group consisting of 4xHis, 5xHis, 6xHis, 7xHis, 8xHis, and mixtures thereof.

3. The composition of claim 1, wherein the affinity tag comprises a C-terminal 6xHIS tag.

4. An isolated and purified S. aureus peptide deformylase iron complex, wherein the complex retains greater than about 95% of its catalytic deformylase activity after being stored at a protein concentration of greater than about 10 mg/ml in 50% glycerol at −20° C. for six months.

5. The complex of claim 4, wherein the complex has an N-terminal or a C-terminal affinity tag.

6. The composition of claim 5, wherein the affinity tag is selected from the group consisting of 4xHis, 5xHis, 6xHis, 7xHis, 8xHis, and mixtures thereof.

7. The composition of claim 5, wherein the affinity tag comprises a C-terminal 6xHIS tag.

8. A method of isolating a stable S. aureus iron-liganded peptide deformylase complex comprising preparing a complex with an N-terminal or a C-terminal affinity tag and isolating the tagged complex using immobilized-metal affinity chromatography or metal-chelate affinity chromatography.

9. The method of claim 8, wherein the N-terminal or C-terminal affinity tagged complex is isolated by being bound to a chromatography medium.

10. The method of claim 8, wherein the affinity tag is selected from the group consisting of 4xHis, 5xHis, 6xHis, 7xHis, 8xHis, and mixtures thereof.

11. The method of claim 8, wherein the affinity tag comprises a C-terminal 6xHIS tag.

12. The method of claim 9, wherein the affinity chromatography medium is Ni-NTA or Ni-IDA.

13. The method of claim 8, wherein the complex is purified using ion exchange chromatography.

14. A method of screening for compounds that interact with a peptide deformylase comprising introducing the compound into a system including a stable S. aureus iron-peptide deformylase complex and measuring for inhibition of peptide deformylase activity.

15. The method of claim 14, wherein the inhibition is measured by a direct or coupled assay method.

16. The method of claim 14, wherein the system is substantially free of catalase.

17. The method of claim 14, wherein the system is substantially free of antioxidants.
